(19) United States
(12) Patent Application Publication
(10) Pub. No.: US 2012/0058074 A1
(43) Pub. Date: Mar. 8, 2012

(54) Deodorizing Compositions

(75) Inventors: Volker Braig, Weinheim-Lutzelsachsen (DE); Thomas Daniel, Waldsee (DE)

(73) Assignee: BASF SE, Ludwigshafen (DE)

(21) Appl. No.: 13/319,428

(22) PCT Filed: May 10, 2010
(86) PCT No.: PCT/EP2010/056313

§ 371 Date: Nov. 8, 2011

(30) Foreign Application Priority Data
May 14, 2009 (EP) 09160285.4
Jul. 13, 2009 (EP) 09165278.4

(51) Int. Cl. A61L 11/00

(52) U.S. Cl. 424/76.6

(57) ABSTRACT

The present invention relates to odor-inhibiting compositions comprising water-absorbing polymer particles and at least one oxidase.

The present invention relates to odor-inhibiting compositions comprising water-absorbing polymer particles and at least one oxidase.

Water-absorbing polymer particles are used to produce diapers, tampons, sanitary napkins and other hygiene articles, but also as water-retaining agents in market gardening. The water-absorbing polymer particles are also referred to as superabsorbents.

The production of water-absorbing polymer particles is described in the monograph “Modern Superabsorbent Polymer Technology”, F. L. Buchholz and A. T. Graham, Wiley-VCH, 1998, pages 71 to 103.

The properties of the water-absorbing polymer particles can be adjusted, for example, via the amount of crosslinker used. With increasing amount of crosslinker, the centrifuge retention capacity (CRC) falls and the absorption under a pressure of 21.0 g/cm² (AUL0.3 psi) passes through a maximum.

To improve the application properties, for example permeability of the swollen gel bed (SFC) in the diaper and absorption under a pressure of 49.2 g/cm² (AUL0.7 psi), water-absorbing polymer particles are generally surface postcrosslinked. This increases the degree of crosslinking of the particle surface, which allows the absorption under a pressure of 49.2 g/cm² (AUL0.7 psi) and the centrifuge retention capacity (CRC) to be at least partly decoupled. This surface postcrosslinking can be performed in the aqueous gel phase. Preferably, however, dried, ground and sieved-off polymer particles (base polymer) are surface coated with a surface postcrosslinker, thermally surface postcrosslinked and dried. Crosslinkers suitable for this purpose are compounds which can form covalent bonds with at least two carboxylate groups of the water-absorbing polymer particles.

WO 99/08726 A1 teaches the use of combinations of haloperoxidases and hydrogen peroxide sources for odor inhibition.

WO 2004/112851 A1 describes enzyme-containing compositions which can be used, for example, as wound dressings. More particularly, lactate oxidase is intended to degrade lactate present in wounds and prevent overacidification.

WO 2006/078868 A2 discloses a preservative system for foods, consisting of a superabsorbent material and bacterial inhibitors.

It was an object of the present invention to provide an improved process for producing odor-inhibiting water-absorbing polymer particles.

The object was achieved by odor-inhibiting compositions comprising water-absorbing polymer particles and at least one oxidase, said composition being essentially free of peroxidases or the specific catalytic peroxidase activity of the composition being less than 0.001 μmol of substrate g⁻¹·min⁻¹.

In the course of oxidation of the substrate, oxidases transfer electrons released to oxygen to form hydrogen peroxide. According to the systematic nomenclature of the Enzyme Commission of the International Union of Biochemistry (IUB), the oxidases belong to the first enzyme class.

Suitable enzymes are oxidases of group EC 1.1.3.x, such as glucose oxidases, (EC number 1.1.3.4), of group EC 1.3.3.x, such as bilirubin oxidases (EC number 1.3.3.5), of group EC 1.4.3.x, such as glycine oxidases (EC number 1.4.3.19), of group EC 1.5.3.x, such as polyamine oxidases (EC number 1.5.3.11), of group EC 1.6.3.x, such as NAD(P)H oxidases (EC number 1.6.3.1), of group EC 1.7.3.x, such as hydroxylamine oxidases (EC number 1.7.3.4), of group EC 1.8.3.x, such as sulfite oxidases (EC number 1.8.3.1), of group EC 1.9.3.x, such as cytochrome oxidases (EC number 1.9.3.1), of group 1.10.3.x, such as catechol oxidases (EC number 1.10.3.1), of group EC 1.16.3.x, such as ferroxidase (EC number 1.16.3.1), of group 1.17.3.x, such as xanthine oxidases (EC number 1.17.3.2), and of EC group 1.21.3.z, such as reticuline oxidases (EC number 1.21.3.3).

Advantageously, a glucose oxidase (EC number 1.1.3.4) is used. It is even more advantageous when the glucose oxidase comprises very little catalase (EC number 1.11.1.6), if any at all.

The oxidases can also be used in encapsulated form, such that they are available only on addition of liquid, for example by means of a coating with water-soluble polymers such as polyvinyl alcohol.

The specific catalytic oxidase activity of the odor-inhibiting composition is preferably from 0.01 to 1000 μmol of substrate g⁻¹·min⁻¹, more preferably from 0.1 to 100 μmol of substrate g⁻¹·min⁻¹, most preferably from 1 to 10 μmol of substrate g⁻¹·min⁻¹.

The specific catalytic oxidase activity of the composition can be determined by customary methods. However, it is better to determine the catalytic activity of the oxidase itself, and to determine the specific catalytic oxidase activity of the composition by calculation.

The odor-inhibiting compositions may additionally also comprise the substrate of the oxidase. A substrate is a compound which is converted by the enzyme in a chemical reaction. The first step of an enzymatic reaction consists in the formation of an enzyme-substrate complex which, after the reaction, leads to the release of product and enzyme, such that the catalysis cycle can be passed through again. An enzyme may possibly convert several different substrates which are often chemically similar. Substrates in the context of the present invention are substrates of the oxidases usable in accordance with the invention, for example β-D-glucose for glucose oxidase.

Preferably from 0.5 to 25% by weight, more preferably from 5 to 20% by weight and most preferably from 8 to 15% by weight of the substrate is used, based in each case on the water-absorbing polymer particles.

The substrates can also be used in encapsulated form, such that they are only available on addition of liquid to the oxidase, for example by means of a coating with water-soluble polymers such as polyvinyl alcohol. It is also possible instead or in addition to encapsulate the oxidases for use in accordance with the invention.

The present invention is based on the finding that oxidases can reduce unpleasant odors in hygiene articles, especially unpleasant odors caused by sulfur compounds. This is possibly achieved by hydrogen peroxide obtained owing to the catalytic oxidase activity. Peroxidases decompose hydrogen peroxide. Therefore, the simultaneous use of peroxidases should be avoided.

The inventive composition preferably comprises at least 90% by weight, more preferably at least 95% by weight, most preferably at least 97% by weight, of water-absorbing polymer particles.

The water-absorbing polymer particles preferably comprise at least 50% by weight of polymerized acrylic acid or salts thereof. Moreover, the water-absorbing polymer particles have preferably been surface postcrosslinked.

In a preferred embodiment of the present invention, the water-absorbing polymer particles have been coated with a zinc salt.

Suitable zinc salts are, for example, zinc hydroxide, zinc sulfate, zinc chloride, zinc citrate, zinc acetate and zinc lactate. Preference is given to using zinc salts of fatty acids, for example of ricinoleic acid.

The amount of zinc salt used is preferably 0.01 to 5% by weight, more preferably 0.05 to 3% by weight, most preferably 0.1 to 1% by weight, based in each case on the water-absorbing polymer.

The zinc salts are typically used as a solution in a suitable solvent, preferably water. Additional coating of the polymer particles with zinc salts can have a favorable influence on the discoloration tendency of the water-absorbing polymer particles.

The production of the water-absorbing polymer particles will be explained in detail hereinafter.

The water-absorbing polymer particles are produced, for example, by polymerizing a monomer solution or suspension comprising
a) at least one ethylenically unsaturated monomer which bears acid groups and may be at least partly neutralized,
b) at least one crosslinker,
c) at least one initiator,
d) optionally one or more ethylenically unsaturated monomers copolymerizable with the monomers mentioned under a) and
e) optionally one or more water-soluble polymers,
and are typically water-insoluble.

The monomers a) are preferably water-soluble, i.e. the solubility in water at 23° C. is typically at least 1 g/100 g of water, preferably at least 5 g/100 g of water, more preferably at least 25 g/100 g of water, most preferably at least 35 g/100 g of water.

Suitable monomers a) are, for example, ethylenically unsaturated carboxylic acids, such as acrylic acid, methacrylic acid and itaconic acid. Particularly preferred monomers are acrylic acid and methacrylic acid. Very particular preference is given to acrylic acid.

Further suitable monomers a) are, for example, ethylenically unsaturated sulfonic acids, such as styrenesulfonic acid and 2-acrylamido-2-methylpropanesulfonic acid (AMPS).

Impurities can have a considerable influence on the polymerization. The raw materials used should therefore have a maximum purity. It is therefore often advantageous to specially purify the monomers a). Suitable purification processes are described, for example, in WO 2002/055469 A1, WO 2003/078378 A1 and WO 2004/035514 A1. A suitable monomer a) is, for example, acrylic acid purified according to WO 2004/035514 A1 comprising 99.8460% by weight of acrylic acid, 0.0950% by weight of acetic acid, 0.0332% by weight of water, 0.0203% by weight of propionic acid, 0.0001% by weight of furfurals, 0.0001% by weight of maleic anhydride, 0.0003% by weight of diacrylic acid and 0.0050% by weight of hydroquinone monomethyl ether.

The proportion of acrylic acid and/or salts thereof in the total amount of monomers a) is preferably at least 50 mol %, more preferably at least 90 mol %, most preferably at least 95 mol %.

The monomers a) typically comprise polymerization inhibitors, preferably hydroquinone monoethers, as storage stabilizers.

The monomer solution comprises preferably up to 250 ppm by weight, preferably at most 130 ppm by weight, more preferably at most 70 ppm by weight, preferably at least 10 ppm by weight, more preferably at least 30 ppm by weight, especially around 50 ppm by weight, of hydroquinone monoether, based in each case on the unneutralized monomer a). For example, the monomer solution can be prepared by using an ethylenically unsaturated monomer bearing acid groups with an appropriate content of hydroquinone monoether.

Preferred hydroquinone monoethers are hydroquinone monomethyl ether (MEHQ) and/or alpha-tocopherol (vitamin E).

Suitable crosslinkers b) are compounds having at least two groups suitable for crosslinking. Such groups are, for example, ethylenically unsaturated groups which can be polymerized free-radically into the polymer chain, and functional groups which can form covalent bonds with the acid groups of the monomer a). In addition, polyvalent metal salts which can form coordinate bonds with at least two acid groups of the monomer a) are also suitable as crosslinkers b).

Crosslinkers b) are preferably compounds having at least two polymerizable groups which can be polymerized free-radically into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1, di- and triacrylates, as described in EP 0 547 847 A1, EP 0 559 476 A1, EP 0 632 068 A1, WO 93/21237 A1, WO 2003/104299 A1, WO 2003/104300 A1, WO 2003/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 2002/032962 A2.

Preferred crosslinkers b) are pentaerythrityl triallyl ether, tetraalloxyethane, methylenebismethacrylamide, 15-tuply ethoxylated trimethylolpropane triacrylate, polyethylene glycol diacrylate, trimethylolpropane triacrylate and triallylamine.

Very particularly preferred crosslinkers b) are the polyethoxylated and/or -propoxylated glycerols which have been esterified with acrylic acid or methacrylic acid to give di- or triacrylates, as described, for example, in WO 2003/104301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol, especially the triacrylate of 3-tuply ethoxylated glycerol.

The amount of crosslinker b) is preferably 0.05 to 1.5% by weight, more preferably 0.1 to 1% by weight, most preferably 0.3 to 0.6% by weight, based in each case on monomer a). With rising crosslinker content, the centrifuge retention capacity (CRC) falls and the absorption under a pressure of 21.0 g/cm² passes through a maximum.

The initiators c) used may be all compounds which generate free radicals under the polymerization conditions, for example thermal initiators, redox initiators, photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is, however, preferably a mixture of the sodium salt of 2-hydroxy-2-sulfinatoacetic acid, the disodium salt of 2-hydroxy-2-sulfonatoacetic acid and sodium bisulfite. Such mixtures are obtainable as Brüggolite® FF6 and Brüggolite® FF7 (Brüggemann Chemicals; Heilbronn; Germany).

Ethylenically unsaturated monomers d) copolymerizable with the ethylenically unsaturated monomers a) bearing acid groups are, for example, acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate.

The water-soluble polymers e) used may be polyvinyl alcohol, polyvinylpyrrolidone, starch, starch derivatives, modified cellulose, such as methylcellulose or hydroxyethylcellulose, gelatin, polyglycols or polyacrylic acids, preferably starch, starch derivatives and modified cellulose.

Typically, an aqueous monomer solution is used. The water content of the monomer solution is preferably from 40 to 75% by weight, more preferably from 45 to 70% by weight, most preferably from 50 to 65% by weight. It is also possible to use monomer suspensions, i.e. monomer solutions with excess monomer a), for example sodium acrylate. With rising water content, the energy requirement in the subsequent drying rises, and, with falling water content, the heat of polymerization can only be removed inadequately.

For optimal action, the preferred polymerization inhibitors require dissolved oxygen. The monomer solution can therefore be freed of dissolved oxygen before the polymerization by inertization, i.e. flowing an inert gas through, preferably nitrogen or carbon dioxide. The oxygen content of the monomer solution is preferably lowered before the polymerization to less than 1 ppm by weight, more preferably to less than 0.5 ppm by weight, most preferably to less than 0.1 ppm by weight.

Suitable reactors are, for example, kneading reactors or belt reactors. In the kneader, the polymer gel formed in the polymerization of an aqueous monomer solution or suspension is comminuted continuously by, for example, contrarotatory stirrer shafts, as described in WO 2001/038402 A1. Polymerization on a belt is described, for example, in DE 38 25 366 A1 and U.S. Pat. No. 6,241,928. Polymerization in a belt reactor forms a polymer gel, which has to be comminuted in a further process step, for example in an extruder or kneader.

However, it is also possible to dropletize an aqueous monomer solution and to polymerize the droplets obtained in a heated carrier gas stream. This allows the process steps of polymerization and drying to be combined, as described in WO 2008/040715 A2 and WO 2008/052971 A1.

The acid groups of the resulting polymer gels have typically been partially neutralized. Neutralization is preferably carried out at the monomer stage. This is typically done by mixing in the neutralizing agent as an aqueous solution or preferably also as a solid. The degree of neutralization is preferably from 25 to 85 mol %, for “acidic” polymer gels more preferably from 30 to 60 mol %, most preferably from 35 to 55 mol %, and for “neutral” polymer gels more preferably from 65 to 80 mol %, most preferably from 70 to 75 mol %, for which the customary neutralizing agents can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogencarbonate and also mixtures thereof.

However, it is also possible to carry out neutralization after the polymerization, at the stage of the polymer gel formed in the polymerization. It is also possible to neutralize up to 40 mol %, preferably 10 to 30 mol % and more preferably 15 to 25 mol % of the acid groups before the polymerization by adding a portion of the neutralizing agent actually to the monomer solution and setting the desired final degree of neutralization only after the polymerization, at the polymer gel stage. When the polymer gel is neutralized at least partly after the polymerization, the polymer gel is preferably comminuted mechanically, for example by means of an extruder, in which case the neutralizing agent can be sprayed, sprinkled or poured on and then carefully mixed in. To this end, the gel mass obtained can be repeatedly extruded for homogenization.

The polymer gel is then preferably dried with a belt drier until the residual moisture content is preferably 0.5 to 15% by weight, more preferably 1 to 10% by weight, most preferably 2 to 8% by weight, the residual moisture content being determined by EDANA (European Disposables and Nonwovens Association) recommended test method No. WSP 230.2-05 “Moisture Content”. In the case of too high a residual moisture content, the dried polymer gel has too low a glass transition temperature T_{g} and can be processed further only with difficulty. In the case of too low a residual moisture content, the dried polymer gel is too brittle and, in the subsequent comminution steps, undesirably large amounts of polymer particles with an excessively low particle size (fines) are obtained. The solids content of the gel before the drying is preferably from 25 to 90% by weight, more preferably from 35 to 70% by weight, most preferably from 40 to 60% by weight. Optionally, it is, however, also possible to use a fluidized bed drier or a paddle drier for the drying operation.

Thereafter, the dried polymer gel is ground and classified, and the apparatus used for grinding may typically be single- or multistage roll mills, preferably two- or three-stage roll mills, pin mills, hammer mills or vibratory mills.

The mean particle size of the polymer particles removed as the product fraction is preferably at least 200 μm, more preferably from 250 to 600 μm, very particularly from 300 to 500 μm. The mean particle size of the product fraction may be determined by means of EDANA (European Disposables and Nonwovens Association) recommended test method No. WSP 220.2-05 “Particle Size Distribution”, where the proportions by mass of the screen fractions are plotted in cumulative form and the mean particle size is determined graphically. The mean particle size here is the value of the mesh size which gives rise to a cumulative 50% by weight.

The proportion of particles with a particle size of at least 150 μm is preferably at least 90% by weight, more preferably at least 95% by weight, most preferably at least 98% by weight.

Polymer particles with too small a particle size lower the permeability (SFC). The proportion of excessively small polymer particles (fines) should therefore be small.

Excessively small polymer particles are therefore typically removed and recycled into the process. This is preferably done before, during or immediately after the polymerization, i.e. before the drying of the polymer gel. The excessively small polymer particles can be moistened with water and/or aqueous surfactant before or during the recycling.

It is also possible in later process steps to remove excessively small polymer particles, for example after the surface postcrosslinking or another coating step. In this case, the excessively small polymer particles recycled are surface postcrosslinked or coated in another way, for example with fumed silica.

When a kneading reactor is used for polymerization, the excessively small polymer particles are preferably added during the last third of the polymerization. When the excessively small polymer particles are added at a very early stage, for example actually to the monomer solution, this lowers the centrifuge retention capacity (CRC) of the resulting water-absorbing polymer particles. However, this can be compensated, for example, by adjusting the amount of crosslinker b) used.

When the excessively small polymer particles are added at a very late stage, for example not until an apparatus connected downstream of the polymerization reactor, for example to an extruder, the excessively small polymer particles can be incorporated into the resulting polymer gel only with difficulty. Insufficiently incorporated, excessively small polymer particles are, however, detached again from the dried polymer gel during the grinding, are therefore removed again in the course of classification and increase the amount of excessively small polymer particles to be recycled.

The proportion of particles having a particle size of at most 850 μm is preferably at least 90% by weight, more preferably at least 95% by weight, most preferably at least 98% by weight.

The proportion of particles having a particle size of at most 600 μm is preferably at least 90% by weight, more preferably at least 95% by weight, most preferably at least 98% by weight.

Polymer particles with too great a particle size lower the swell rate. The proportion of excessively large polymer particles should therefore likewise be small.

Excessively large polymer particles are therefore typically removed and recycled into the grinding of the dried polymer gel.

To further improve the properties, the polymer particles can be surface postcrosslinked. Suitable surface postcrosslinkers are compounds which comprise groups which can form covalent bonds with at least two carboxylate groups of the polymer particles. Suitable compounds are, for example, polyfunctional amines, polyfunctional amidoamines, polyfunctional epoxides, as described in EP 0 083 022 A2, EP 0 543 303 A1 and EP 0 937 736 A2, di- or polyfunctional alcohols, as described in DE 33 14 019 A1, DE 35 23 617 A1 and EP 0 450 922 A2, or β-hydroxyalkylamides, as described in DE 102 04 938 A1 and U.S. Pat. No. 6,239,230.

Additionally described as suitable surface postcrosslinkers are cyclic carbonates in DE 40 20 780 C1, 2-oxazolidone and its derivatives, such as 2-hydroxyethyl-2-oxazolidone in DE 198 07 502 A1, bis- and poly-2-oxazolidinones in DE 198 07 992 C1, 2-oxotetrahydro-1,3-oxazine and its derivatives in DE 198 54 573 A1, N-acyl-2-oxazolidones in DE 198 54 574 A1, cyclic ureas in DE 102 04 937 A1, bicyclic amide acetals in DE 103 34 584 A1, oxetanes and cyclic ureas in EP 1 199 327 A2 and morpholine-2,3-dione and its derivatives in WO 2003/031482 A1.

Preferred surface postcrosslinkers are ethylene carbonate, ethylene glycol diglycidyl ether, reaction products of polyamides with epichlorohydrin, and mixtures of propylene glycol and 1,4-butanediol.

Very particularly preferred surface postcrosslinkers are 2-hydroxyethyloxazolidin-2-one, oxazolidin-2-one and 1,3-propanediol.

In addition, it is also possible to use surface postcrosslinkers which comprise additional polymerizable ethylenically unsaturated groups, as described in DE 37 13 601 A1.

The amount of surface postcrosslinkers is preferably 0.001 to 2% by weight, more preferably 0.02 to 1% by weight, most preferably 0.05 to 0.2% by weight, based in each case on the polymer particles.

In a preferred embodiment of the present invention, polyvalent cations are applied to the particle surface in addition to the surface postcrosslinkers before, during or after the surface postcrosslinking.

The polyvalent cations usable in the process according to the invention are, for example, divalent cations such as the cations of zinc, magnesium, calcium, iron and strontium, trivalent cations such as the cations of aluminum, iron, chromium, rare earths and manganese, tetravalent cations such as the cations of titanium and zirconium. Possible counterions are, for example, chloride, bromide, sulfate, hydrogensulfate, carbonate, hydrogencarbonate, nitrate, phosphate, hydrogenphosphate, dihydrogenphosphate and carboxylate, such as acetate and lactate. Aluminum sulfate and aluminum lactate are preferred. Apart from metal salts, it is also possible to use polyamines as polyvalent cations.

The amount of polyvalent cation used is, for example, 0.001 to 1.5% by weight, preferably 0.005 to 1% by weight, more preferably 0.02 to 0.8% by weight, based in each case on the polymer particles.

The surface postcrosslinking is typically performed in such a way that a solution of the surface postcrosslinker is sprayed onto the dried polymer particles. After the spraying, the polymer particles coated with surface postcrosslinker are dried thermally, and the surface postcrosslinking reaction can take place either before or during the drying. The spraying of a solution of the surface postcrosslinker is preferably performed in mixers with moving mixing tools, such as screw mixers, disk mixers and paddle mixers. Particular preference is given to horizontal mixers such as paddle mixers, very particular preference to vertical mixers. The distinction between horizontal mixers and vertical mixers is made by the position of the mixing shaft, i.e. horizontal mixers have a horizontally mounted mixing shaft and vertical mixers a vertically mounted mixing shaft. Suitable mixers are, for example, horizontal Pflugschar® plowshare mixers (Gebr. Lödige Maschinenbau GmbH; Paderborn; Germany), Vrieco-Nauta continuous mixers (Hosokawa Micron BV; Doetinchem; the Netherlands), Processall Mixmill mixers (Processall Incorporated; Cincinnati; US) and Schugi Flexomix® (Hosokawa Micron BV; Doetinchem; the Netherlands). However, it is also possible to spray on the surface postcrosslinker solution in a fluidized bed.

The surface postcrosslinkers are typically used in the form of an aqueous solution. The content of nonaqueous solvent and/or total amount of solvent can be used to adjust the penetration depth of the surface postcrosslinker into the polymer particles.

When exclusively water is used as the solvent, a surfactant is advantageously added. This improves the wetting performance and reduces the tendency to form lumps. However, preference is given to using solvent mixtures, for example isopropanol/water, 1,3-propanediol/water and propylene glycol/water, where the mixing ratio by mass is preferably from 20:80 to 40:60.

The thermal drying is preferably carried out in contact driers, more preferably paddle driers, most preferably disk driers. Suitable driers are, for example, Hosokawa Bepex® horizontal paddle driers (Hosokawa Micron GmbH; Leingarten; Germany), Hosokawa Bepex® disk driers (Hosokawa Micron GmbH; Leingarten; Germany) and Nara paddle driers (NARA Machinery Europe; Frechen; Germany). Moreover, it is also possible to use fluidized bed driers.

The drying can be effected in the mixer itself, by heating the jacket or blowing in warm air. Equally suitable is a downstream drier, for example a shelf drier, a rotary tube oven or a heatable screw. It is particularly advantageous to mix and dry in a fluidized bed drier.

Preferred drying temperatures are in the range of 100 to 250° C., preferably 120 to 220° C., more preferably 130 to 210° C., most preferably 150 to 200° C. The preferred residence time at this temperature in the reaction mixer or drier is preferably at least 10 minutes, more preferably at least 20 minutes, most preferably at least 30 minutes, and typically at most 60 minutes.

Subsequently, the surface postcrosslinked polymer particles can be classified again, excessively small and/or excessively large polymer particles being removed and recycled into the process.

To further improve the properties, the surface postcrosslinked polymer particles can be coated or remoisturized.

The remoisturizing is carried out preferably at 30 to 80° C., more preferably at 35 to 70° C. and most preferably at 40 to 60° C. At excessively low temperatures, the water-absorbing polymer particles tend to form lumps, and, at higher temperatures, water already evaporates noticeably. The amount of water used for remoisturizing is preferably from 1 to 10% by weight, more preferably from 2 to 8% by weight and most preferably from 3 to 5% by weight. The remoisturizing increases the mechanical stability of the polymer particles and reduces their tendency to static charging.

Suitable coatings for improving the swell rate and the permeability (SFC) are, for example, inorganic inert substances, such as water-insoluble metal salts, organic polymers, cationic polymers and di- or polyvalent metal cations. Suitable coatings for dust binding are, for example, polyols. Suitable coatings for counteracting the undesired caking tendency of the polymer particles are, for example, fumed silica, such as Aerosil® 200, and surfactants, such as Span® 20.

The water-absorbing polymer particles have a moisture content of preferably 1 to 15% by weight, more preferably 2 to 10% by weight, most preferably 3 to 5% by weight, the moisture content being determined by EDANA (European Disposables and Nonwovens Association) recommended test method No. WSP 230.2-05 “Moisture Content”.

The water-absorbing polymer particles have a centrifuge retention capacity (CRC) of typically at least 15 g/g, preferably at least 20 g/g, preferentially at least 22 g/g, more preferably at least 24 g/g, most preferably at least 26 g/g. The centrifuge retention capacity (CRC) of the water-absorbing polymer particles is typically less than 60 g/g. The centrifuge retention capacity (CRC) is determined by EDANA (European Disposables and Nonwovens Association) recommended test method No. WSP 241.2-05 “Centrifuge Retention Capacity”.

The water-absorbing polymer particles have an absorption under a pressure of 49.2 g/cm² of typically at least 15 g/g, preferably at least 20 g/g, preferentially at least 22 g/g, more preferably at least 24 g/g, most preferably at least 26 g/g. The absorption under a pressure of 49.2 g/cm² of the water-absorbing polymer particles is typically less than 35 g/g. The absorption under a pressure of 49.2 g/cm² is determined analogously to EDANA (European Disposables and Nonwovens Association) recommended test method No. WSP 242.2-05 “Absorption under Pressure”, except that a pressure of 49.2 g/cm² is established instead of a pressure of 21.0 g/cm².

The present invention further provides processes for producing the inventive compositions by
i) mixing at least one oxidase together with water-absorbing polymer particles and/or
ii) grinding at least one oxidase together with water-absorbing polymer particles and/or
iii) spraying at least one oxidase onto water-absorbing polymer particles and
iv) optionally mixing the composition obtained in i), ii) and/or iii) together with water-absorbing polymer particles.

Variants i) and iii) are preferred.

In a preferred embodiment of the present invention, the water-absorbing polymer particles are additionally mixed with the substrate of the oxidase, wherein variants i) to iv) are likewise suitable.

The type of mixing is not subject to any restriction and can be effected as early as in the course of production of the water-absorbing polymer particles, for example in the course of cooling after the surface postcrosslinking or the subsequent classifying, or in a specific mixer. Suitable mixers have already been described above for the surface postcrosslinking of the water-absorbing polymer particles.

The type of grinding is likewise not subject to any restriction. Suitable apparatuses have already been described above for the comminution of the water-absorbing polymer particles.

The type of spraying is not subject to any restriction.

In a further preferred embodiment of the present invention, an inventive composition A) comprising water-absorbing polymer particles and at least one oxidase, and a further composition B) comprising water-absorbing polymer particles and at least one substrate of the at least one oxidase, are prepared separately and then mixed.

The mixing ratio of composition A) to composition B) is preferably from 0.01 to 100, more preferably from 0.1 to 10, most preferably from 0.5 to 2.

The type of mixing is not subject to any restriction.

In the production of powder mixtures from water-absorbing polymer particles, at least one oxidase and optionally at least one substrate, antidusting agents are advantageously used. Suitable antidusting agents are polyglycerols, polyethylene glycols, polypropylene glycols, random or block copolymers of ethylene oxide and propylene oxide. Further antidusting agents suitable for this purpose are the polyethoxylates or polypropoxylates of polyhydroxyl compounds, such as glycerol, sorbitol, trimethylolpropane, trimethylolethane and pentaerythritol. Examples thereof are n-tuply ethoxylated trimethylolpropane or glycerol, where n is an integer from 1 to 100. Further examples are block copolymers such as trimethylolpropane or glycerol which have been n-tuply ethoxylated and then m-tuply propoxylated overall, where n is an integer from 1 to 40 and m is an integer from 1 to 40. The sequence of the blocks may also be reversed.

The oxidase can be used as an untreated extract or in concentrated form. It is also possible to use immobilized oxidases on a support. Suitable supports are, for example, clay minerals, bentonites, silica gels, flour, cellulose, water-insoluble phosphates, carbonates or sulfates, and cationic, nonionic or anionic polymers, activated carbon, aluminum oxides, titanium dioxide, fumed silica. The supports may be either granular or fibrous. The binding to the support may be covalent or absorptive.

In a further embodiment, an inventive composition which has a relatively high specific catalytic peroxidase activity is produced, typically 1 to 10 000 μmol of substrate g⁻¹·m⁻¹, preferably 5 to 5000 μmol of substrate g⁻¹·m⁻¹, more preferably 10 to 1000 μmol of substrate g⁻¹·m⁻¹. The highly concentrated composition thus obtained can then be diluted to the desired final content with further water-absorbing polymer particles.

In a further preferred embodiment, a mixture of at least two compositions based on water-absorbing polymer particles is prepared, one of which comprises the enzyme and the other the substrate. The mixing ratio may vary from 1:99 up to 99:1. Particular preference is given to a mixture of water-absorbing polymer particles of similar size.

The type of mixing is not subject to any restriction, and can be effected as early as in the course of preparation of one of the two compositions, for example in the course of cooling after surface postcrosslinking, the subsequent classification, or in a specific mixer. Suitable mixers have already been described above for the surface postcross-linking of the water-absorbing polymer particles.

The present invention further provides hygiene articles comprising at least one inventive composition, and hygiene articles comprising water-absorbing polymer particles, at least one oxidase and a substrate of the oxidase, the oxidase being essentially free of peroxidases or the specific catalytic peroxidase activity of the oxidase being less than 0.001 μmol of substrate g⁻¹·min⁻¹, especially hygiene articles for feminine hygiene, hygiene articles for light and heavy incontinence, or small animal litter.

The hygiene articles typically comprise a water-impervious backside, a water-pervious topside and, in between, an absorbent core of the inventive water-absorbing polymer particles and cellulose fibers. The proportion of the inventive water-absorbing polymer particles in the absorbent core is preferably 20 to 100% by weight, preferentially 50 to 100% by weight.

The inventive hygiene articles may also comprise the substrate of the appropriate oxidase outside the inventive composition. In this case, the substrate is transported to the oxidase only on liquid loading.

In addition, the water-absorbing polymer particles, the oxidase and the substrate can also be introduced separately into the absorbent core.

The water-absorbing polymer particles are tested by means of the test methods described below.

### Methods:

The measurements should, unless stated otherwise, be carried out at an ambient temperature of 23±2° C. and a relative air humidity of 50±10%. The water-absorbing polymer particles are mixed thoroughly before the measurement.

### Centrifuge Retention Capacity

The centrifuge retention capacity (CRC) is determined by EDANA recommended test method No. WSP 241.2-05 “Centrifuge Retention Capacity”.

### Bacteria-Induced Ammonia Release

DSM1 medium (Deutsche Sammlung von Mikroorganismen and Zellkulturen GmbH) was prepared from 5.0 g/l of peptone from meat (Merck KGaA; Darmstadt; Germany; Art. No. 1.07214) and 3.0 g/l of meat extract (Merck KGaA, Darmstadt; Germany; Art. No. 1103979) and adjusted to pH=7.0. 50 ml of DSM1 medium were inoculated to OD=0.1 with Proteus mirabilis ATCC 14153, and incubated in a 250 ml baffled Erlenmeyer flask at 37° C. and 220 rpm for 15 hours. The cultures thus produced had a cell density of about 10⁹ CFU/ml (OD=2.0-2.5).

The synthetic urine was prepared from 25 g/l of urea (sterile-filtered), 9.0 g/l of sodium chloride, 0.5 g/l of β-D-glucose, 1 g/l of peptone from meat and 1 g/l of meat extract. The synthetic urine was autoclaved before addition of a sterile-filtered concentrated urea solution.

125 ml polypropylene histology beakers were autoclaved, and the amount of water-absorbing polymer particles needed to absorb 50 ml of synthetic urine was introduced (calculated from the centrifuge retention capacity). Then 50 ml of synthetic urine were inoculated with 50 μl of bacterial strain solution corresponding to a total concentration of approx. 10⁶ CFU/ml and mixed with the water-absorbing polymer particles, and the lid provided with a diffusion test tube (Dragerwerk AG & Co. KGaA; Lübeck; Germany; Dräger Tube® Ammonia 20/a-D; Art. No. 8101301) was screwed on immediately. The evolution of ammonia was observed at 37° C. over 48 hours.

### EXAMPLES

The examples were carried out with Hysorb® B7065 (BASF SE; Ludwigshafen; Germany), commercial surface postcrosslinked water-absorbing polymer particles based on sodium acrylate with a degree of neutralization of 70 to 75 mol %.

Such surface postcrosslinked water-absorbing polymer particles are commercially available, for example, from BASF Aktiengesellschaft (trade name: HySorb®), from Stockhausen GmbH (trade name: Favor®) and from Nippon Shokubai Co., Ltd. (trade name: Aqualic®).

### Example 1

20 g of water-absorbing polymer particles were weighed into a 50 ml glass bottle with 0.02 g of “Gluzyme® Mono 10000 BG” (Novozymes A/S; Bagsvaerd; Denmark). “Gluzyme® Mono 10000 BG” is a glucose oxidase with a specific catalytic activity of 10 000 μmol of substrate g⁻¹·min⁻¹. Subsequently, the mixture was transferred into a large porcelain mortar (internal diameter 16 cm) and triturated there for approx. 5 minutes. In addition, the samples were homogenized once again in a tumbling mixer at 46 rpm for 20 minutes.

### Example 2

0.33 g of “Multifect® GO 5000L” (Genencor International B.V.; Leiden; The Netherlands) was weighed into a 25 ml penicillin bottle and made up to 10 g with ultrapure water. “Multifect® GO 5000L” is a glucose oxidase with a specific catalytic activity of 4000 μmol of substrate g⁻¹·min⁻¹.

20.0 g of water-absorbing polymer particles were introduced into a modified coffee grinder (Blender 8012 Model 34BL99; Waring Laboratory; US) with a stainless steel attachment (internal diameter 8 cm, internal height 4 cm, tool diameter 7 cm, addition point in the lid 1.3 cm away from the edge, baffles in the lid). The modified coffee grinder was operated at level 3. A syringe with a cannula was used to add 0.60 g of the enzyme solution slowly. After the addition had ended, the water-absorbing polymer particles were transferred into a glass dish and left to stand at room temperature in a fume hood for 30 minutes.

### Example 3

The procedure was as in example 2, except that 0.33 g of “GC 199 Enzyme Preparation” (Genencor International B.V.; Leiden; The Netherlands) was weighed into a 25 ml penicillin bottle and made up to 10 g with ultrapure water. “GC 199 Enzyme Preparation” is a glucose oxidase with a specific catalytic activity of 1500 μmol of substrate g⁻¹·min⁻¹.

### Example 4

The procedure was as in example 1, except that the water-absorbing polymer particles were blended beforehand with 12.8% by weight of β-D-glucose and, in the preparation of the synthetic urine, the β-D-glucose was omitted.

### Example 5

3.4 g of potassium hydrogenphosphate were weighed in and made up to 250 ml with deionized water. 5.7 g of dipotassium hydrogenphosphate were weighed into a second standard flask and likewise made up to 250 ml. Subsequently, a sufficient amount of potassium hydrogenphosphate solution was added to the dipotassium hydrogen-phosphate solution so that a pH of 7 was attained (buffer solution).

267 mg of “Gluzyme® Mono 10000 BG” (Novozymes NS; Bagsvaerd; Denmark) were slurried in 80 g of buffer solution (enzyme solution).

270 g of water-absorbing polymer particles were introduced into a Bosch MultiMixx47 of the CNUM5EV model (Robert Bosch Hausgeräte GmbH; Munich, Germany) with a 3-bar beater and stirred at level 3. 8.1 g of the enzyme solution were sprayed on with a spray atomizer (800 l/h of nitrogen). This was followed by stirring at level 1 for a further 10 minutes and, after the material covering the wall had been removed with a brush, stirring at level 1 for another 10 minutes.

50 g of this mixture were triturated with 1 g of β-D-glucose in a porcelain mortar (internal diameter 16 cm) and then homogenized in a tumbling mixer at 32 rpm for 30 minutes. In the preparation of the synthetic urine for the bacteria-induced ammonia release, the β-D-glucose was omitted.

TABLE 1
Test results

| | CRC | Time until attainment of 1500 ppm · h | |
|---|---|---|---|
| Example | [g/g] | of ammonia or value after 48 h | |
| Hysorb ® B7065 | 29.8 | 8.75 | h |
| 1 | 30.1 | ammonia no longer detectable | |
| 2 | 29.0 | 30 | ppm · h |
| 3 | 29.3 | 45 | ppm · h |

| | | |
|---|---|---|
| 4 | 26.4 | ammonia no longer detectable |
| 5 | 26.9 | ammonia no longer detectable |

1. An odor-inhibiting composition comprising water-absorbing polymer particles and at least one oxidase, said composition being essentially free of peroxidases or a specific catalytic peroxidase activity of the composition being less than 0.001 μmol of substrate g⁻¹·min⁻¹.

2. The composition according to claim 1, which comprises a substrate of the oxidase.

3. The composition according to claim 1, wherein the oxidase is a glucose oxidase.

4. The composition according to claim 1, wherein the oxidase is a glucose oxidase and the composition comprises glucose.

5. The composition according to claim 1, wherein the specific catalytic oxidase activity of the composition is from 0.01 to 1000 μmol of substrate g⁻¹·min⁻¹.

6. The composition according to claim 1, wherein the composition comprises at least 90% by weight of water-absorbing polymer particles.

7. The composition according to claim 1, wherein the water-absorbing polymer particles comprise at least 50% by weight of polymerized acrylic acid.

8. The composition according to claim 1, wherein the water-absorbing polymer particles have been surface postcrosslinked.

9. The composition according to claim 1, wherein the water-absorbing polymer particles have a centrifuge retention capacity of at least 15 g/g.

10. A process for producing compositions defined in claim 1, which comprises performing at least one of the following steps:
i) mixing at least one oxidase together with water-absorbing polymer particles and optionally a substrate of the oxidase and/or
ii) grinding at least one oxidase and optionally a substrate of the oxidase together with a water-absorbing polymer and/or
iii) spraying at least one oxidase and optionally the substrate of the oxidase onto water-absorbing polymer particles and
iv) optionally mixing the composition obtained in i), ii) and/or iii) together with further water-absorbing polymer particles.

11. A hygiene article comprising at least one composition according to claim 1.

12. A hygiene article comprising water-absorbing polymer particles, at least one oxidase, and a substrate of the oxidase, said oxidase being essentially free of peroxidases or a specific catalytic peroxidase activity of the oxidase being less than 0.001 μmol of substrate g⁻¹·min⁻¹.

13. The hygiene article according to claim 12, wherein the oxidase is a glucose oxidase and the substrate is glucose.

14. The hygiene article according to claim 12, wherein the water-absorbing polymer particles have a centrifuge retention capacity of at least 15 g/g.

15. The hygiene article according to claim 11, wherein the hygiene article is an article for feminine hygiene, an article for light and heavy incontinence, or small animal litter.
